(19) United States
(12) Patent Application Publication
(10) Pub. No.: US 2011/0142894 A1
(43) Pub. Date: Jun. 16, 2011

(54) DISPERSION CONTAINING ZINC OXIDE OR TITANIUM DIOXIDE

(75) Inventors: Ayaka WATANABE, Iwaki-shi (JP); Takuro ASHIDA, Iwaki-shi (JP)

(73) Assignee: SAKAI CHEMICAL INDUSTRY CO., LTD., Osaka (JP)

(21) Appl. No.: 12/898,302

(22) Filed: Oct. 5, 2010

(30) Foreign Application Priority Data
Oct. 6, 2009 (JP) 2009-232737

(51) Int. Cl. A61K 8/92; A61K 8/02; A61Q 17/04; A61Q 1/02

(52) U.S. Cl. 424/401; 424/642; 424/617; 424/59

(57) ABSTRACT

The present invention has an object to provide a high concentration dispersion of zinc oxide or titanium dioxide which is free of volatile silicones, as well as a cosmetic composition which gives little sticky feel after spreading on the skin. The present invention relates to a dispersion which contains: zinc oxide or titanium dioxide; an isoparaffin having the average polymerization degree of 3 to 10; and a polyether-modified silicone having an HLB value of 2 to 5, as a dispersant; the amount of the zinc oxide or titanium dioxide being not less than 60% by mass of the whole amount of the dispersion.

### FIELD OF THE INVENTION

The present invention provides a dispersion that has a high pigment concentration of zinc oxide or titanium dioxide, and is free of volatile silicones. The dispersion provided by the present invention is useful especially in the field of cosmetics, such as sunscreen or foundation.

### BACKGROUND OF THE INVENTION

Fine particulate zinc oxide and titanium dioxide are known to have ultraviolet absorbing ability originating in their characteristic band gap. Fine particulate zinc oxide or titanium dioxide which, for example, has a primary diameter of about 10 to 100 nm, penetrates visible light (the wave length of which is 400 to 800 nm), but scatters UV ray (the wavelength of which is shorter than the visible light). Therefore, fine particulate zinc oxide and titanium dioxide have been used in a variety of fields. For example, they are used as UV-cut fillers in cosmetics such as sunscreen, or as pigments for plastics, inks, and the like.

Fine particulate zinc oxide and titanium dioxide are easy to agglomerate. Therefore, when powder of such zinc oxide and titanium dioxide are directly blended in cosmetics, transparency and UV-cut ability becomes insufficient. In addition, such cosmetics give unpleasant feel such as rough touch if they are spread over the skin. Therefore, in general, they are first dispersed in a suitable dispersion medium using dispersion machines, such as a bead mill, and then blended in cosmetics or the like compositions.

As such a dispersion medium, a medium drying rapidly after spreading is preferred. Especially in cosmetic fields, volatile silicones, in particular, decamethylcyclopentasiloxane, have been widely used because they gives smooth touch without stickiness, and cause no chilly feeling because of its small heat of vaporization when they are spread on the skin (for example, see Patent Documents 1).

The toxicity of decamethylcyclopentasiloxane is not clear. However, decamethylcyclopentasiloxane is chemically stable, and therefore, Environment Canada and other authorities have raised an issue on persistence of the material in the environment. The issue is not limited to decamethylcyclopentasiloxane. Other volatile silicones are also chemically stable, and they may cause a similar problem on persistence in the environment. Therefore, dispersions that contain other oils as dispersing media in lieu of volatile silicones have been tested.

However, when the other oils are used in place of volatile silicones, a significant quantity of dispersion media and dispersants are required. As a result, pigment concentration in a dispersion cannot be made high enough. In a composition like a cosmetic, it is preferable that the amounts of other components than active ingredients are as little as possible. Thus, a dispersion containing little amount of dispersants and additional components, namely, a dispersion having a high pigment concentration has been required. Specifically, preferable pigment dispersion as a material of cosmetics has a pigment concentration of not less than 60% by mass.

However, some problems may arise on a dispersion in a conventional oil when the pigment concentration is high. For example, such a dispersion with a pigment concentration of as high as not less than 60% by mass is quite low in dispersibility, and still worse, it may form a gel. Thus, it is difficult to prepare a dispersion of pigments (such as fine particulate zinc oxide or titanium dioxide) with a high pigment concentration, which is suitable for a material of cosmetics or the like (see Patent Documents 2 and 3).

In addition, quick driability, which is one of the characteristics of volatile silicone-containing cosmetics, is not exerted if the other oils are used as dispersing media. When the other oils are blended in a cosmetic composition (for example, sunscreens), feel of stickiness may remain on the skin after spreading. Such a problem has not been overcome yet.

Other trials for heightening the pigment concentration has been made, for example, by selecting dispersants, but various problems has been revealed. For example, Patent Document 4 proposes use of a particular reactive organic modified silicone as a dispersant. A silicone having a reactive functional group, however, is unstable, and may denaturalized owing to a reaction between the reactive functional group with titanium dioxide or zinc oxide at the active site of the surface of the oxide, or a reaction between the functional group with an ion eluted from titanium dioxide or zinc oxide. As a result, dispersion stability may deteriorate.

### REFERENCES

### Patent Documents

[Patent document 1] JP 2004-83452 A
[Patent document 2] JP 6-61457 B
[Patent document 3] JP 2006-1886 A
[Patent document 4] JP 2002-80771 A

### BRIEF SUMMARY OF THE INVENTION

In view of the state as mentioned above, the present invention has an object to provide a high concentration dispersion of zinc oxide or titanium dioxide which is free of volatile silicones.

The present inventors have made intensive studies, and as a result, they found that a specific isoparaffin as a dispersion medium, and a polyether-modified silicone with an HLB value of 2 to 5 as a dispersing agent enable to provide a dispersion containing zinc oxide or titanium dioxide in a high concentration. Such a dispersion is excellent in dispersion stability. Additionally, they found that a cosmetic composition made from such a dispersion does not give a sticky feel.

The first aspect of the present invention relates to a dispersion which contains: zinc oxide or titanium dioxide; an isoparaffin having the average polymerization degree of 3 to 10; and a polyether-modified silicone having an HLB value of 2 to 5, as a dispersant; the amount of the zinc oxide or titanium dioxide being not less than 60% by mass of the whole amount of the dispersion.

In this aspect, the amount of the zinc oxide or titanium dioxide is preferably not less than 70% by mass of whole amount of the dispersion.

The dispersion preferably contains 2 to 15% by mass of the polyether-modified silicone relative to the whole amount of the dispersion.

Preferable zinc oxide or titanium dioxide has a surface treated with at least one of the group consisting of silica, alumina, alkyl silanes, and organopolysiloxane. The zinc oxide or titanium dioxide preferably has an average primary particle diameter of 100 nm or smaller.

The second aspect of the present invention relates to a cosmetic composition which contains any one of the above-mentioned dispersions.

The dispersion of the present invention contains an isoparaffin instead of volatile silicones. Thus, the present invention provides a dispersion suitable for, but not limited to, a cosmetic material without using volatile silicones which may cause a problem of persistence in the environment.

In addition, the isoparaffin enables to give a high concentration pigment dispersion, which has been difficult to produce using conventional oils as a dispersion medium. An isoparaffin having the average polymerization degree of 3 to 10 is preferable since it maintains the volatility of a dispersion medium, and a cosmetic composition containing such an isoparaffin brings excellent feeling of use when spread on the skin.

When a polyether-modified silicone having a HLB value of 2 to 5 is used as a dispersion medium, a dispersion of zinc oxide or titanium dioxide which is stable even in a high concentration state can be obtained.

### DETAILED DESCRIPTION OF THE INVENTION

The present invention is explained in detail below.

### (Zinc Oxide and Titanium Dioxide)

The dispersion of the present invention contains zinc oxide or titanium dioxide. Particle size or particle shape of the zinc oxide and titanium dioxide is not limited, and the size or shape of particles may be selected depending on the purpose of the obtained particles. In order to enhance blocking ability to ultraviolet ray and transparency of visible light, an average primary diameter observed by an electron microscope is preferably 100 nm or less, and more preferably 10 to 50 nm. In the case of spindle-shaped titanium dioxide, an average primary diameter herein means the size of the minor axis direction.

The zinc oxide or titanium dioxide used in the present invention may have a surface treated with an inorganic material for controlling particle activity, improvement of feeling of use of cosmetic compositions, hydrophobization of the particle surface. Particularly, zinc oxide or titanium dioxide whose surface is treated with silica, alumina, or aluminum hydroxide is preferred. The zinc oxide or titanium dioxide may also be coated with organic substances or organic polymers for improving wettability of fine particles or for providing water repellency. Particularly, zinc oxide or titanium dioxide coated with an alkylsilane or organopolysiloxane is preferred.

When the surface of the zinc oxide or titanium dioxide is treated with inorganic substances, the coating amount of the inorganic substances is preferably 5 to 60 parts by mass, and more preferably 10 to 45 parts by mass relative to 100 parts by mass of zinc oxide or titanium dioxide. When the amount is less than 5 parts by mass, some effects like the suppressive effect of fine-particle activity may become insufficient, whereas when the amount exceeds 60 parts by mass, the original characteristics of zinc oxide or titanium dioxide may be suppressed.

When the surface of the zinc oxide or titanium dioxide is treated with organic substances or organic polymers, the amount of the organic substances or organic polymers to be used is preferably 1 to 20 parts by mass, and more preferably 2 to 15 parts by mass relative to 100 parts by mass of base materials. When the amount is less than 1 parts by mass, some effects like improving effect of wettability of fine particles or providing effect of water repellency may become insufficient, whereas when the amount exceeds 20 parts by mass, the original characteristics of zinc oxide or titanium dioxide may be interfered. The term “base material” used herein means an object material of the surface treatment with an organic substance or an organic polymer. Here, the base material not only includes zinc oxide and titanium dioxide particle itself. For example, when the zinc oxide and titanium dioxide particle is surface-treated with inorganic materials or the like, the base material means the whole particle including a core material, and coated layers. Thus, the mass of the base material means a total mass of zinc oxide or a titanium dioxide particle and optional surface-treated (inorganic) layer.

### (Isoparaffin)

The dispersion medium used in the present invention is an isoparaffin having the average polymerization degree of 3 to 10. Isoparaffin is also called hydrogenated polyisobutene. Isoparaffin is a mixture of branched hydrocarbons, produced by homopolymerization of isobutene, or copolymerization of isobutene and n-butene, followed by hydrogenation of resultant polymer.

In the present invention, the average polymerization degree of an isoparaffin is 3 to 10, and preferably 4 to 6. Here, the “average polymerization degree of an isoparaffin” means that the total number of isobutene monomer units and n-butene monomer units in a molecule. When the average polymerization degree is smaller than three, the isoparaffin is extremely volatile, and production of dispersion or cosmetics using such an isoparaffin may become difficult, or feeling of use of cosmetics may become worse. On the contrary, when the average polymerization degree exceeds 10, such an isoparaffin may become involatile, and the feeling of use of cosmetics may become worse, too.

The ratio of isobutene monomers and n-butene monomers in the raw material of the isoparaffin is not particularly limited, and may freely be changed according to preference of the feeling of use of product cosmetics, although isobutene monomer units are necessary in the isoparaffin. Among several types of isoparaffins, light isoparaffin is preferred, and more preferred is one which satisfies the standards for light liquid isoparaffin and liquid isoparaffin in Japanese Standards of Quasi-Drug Ingredients 2006 from the viewpoint of safety for human body or economical advantage of quasi drugs. Specific examples include commercially available PerLeam (trade name, NOF corporation), including PerLeam 4 (average polymerization degree: 4 to 6), PerLeam EX (average polymerization degree: 5 to 10), and PerLeam 6 (average polymerization degree: 5 to 10).

### (Dispersant)

The dispersion of the present invention contains a polyether-modified silicone with an HLB value of 2 to 5 as a dispersant.

The HLB (Hydrophilic Lypophilic Balance) value of a dispersant is an index which shows the hydrophilicity/lipophilicity balance of a nonionic surfactant. The larger the HLB value is, the greater the hydrophilicity is, and the smaller the HLB value is, the greater the lipophilicity is. Although some variation are present as the definition of the HLB value, the HLB value herein mentioned are determined according to the following equation proposed by W. C. Grifinn:

NHLB=(E+P)/5

wherein NHLB means a HLB value; E means a percentage (mass basis) of polyoxyethylene portion in the whole molecules in a dispersant, and P means a percentage (mass basis) of polyhydric alcohol portion in the whole molecules in a dispersant.

The polyether-modified silicone is a silicone having a polydimethylpolysiloxane portion and a polyoxyethylene portion. More specifically, the polyether-modified silicone may be a polymer, the main chain of which consists of polydimethylpolysiloxane, and which may have a side chain having a polyoxyethylene portion. Alternatively, the polyether-modified silicone may be a copolymer in which the polydimethylpolysiloxane portion and the polyoxyethylene portion are formed by block copolymerization. As long as the HLB is within the range of 2 to 5, the polyether-modified silicone may be further modified by the other molecular chains, such as alkyl chains, polyhydric alcohol chains, and polyoxypropylene portions. Specific examples of such a polyether-modified silicone include KF-6038 (product of Shin-Etsu chemicals Co., Ltd.).

The amount of polyether-modified silicone is preferably 2 to 15% by mass relative to the whole mass of the dispersion. When the amount of silicone is less than 2% by mass, preparation of dispersion with a high pigment concentration may be difficult, or the storage stability of a dispersion may worsen. If the amount is too large, the degree of freedom on formulation of cosmetics may become narrow, or a feeling of use of cosmetics may worsen.

### (Dispersion)

The dispersion of the present invention contains zinc oxide or titanium dioxide, an isoparaffin having the average polymerization degree of 3 to 10, and a polyether-modified silicone having an HLB value of 2 to 5, as a dispersant. The amount of the zinc oxide or titanium dioxide is not less than 60% by mass of the whole amount of the dispersion.

The amount of zinc oxide or titanium dioxide in the total mass amount of dispersion is preferably not less than 70% by mass. As described above, smaller amount of the dispersion medium or other additives, in other words, the dispersion is preferable to have a high pigment concentration since the amount of components other than active ingredients can be saved, and freedom of formulation of cosmetics, or the like may be wider. However, when other oils are used in place of volatile silicones, there may be a problem that pigment concentration in a dispersion could not be made high enough. The present invention provides a dispersion with a pigment concentration of not less than 60% by mass, which has been difficult to produce if conventional oils are used, by blending an isoparaffin having the average polymerization degree of 3 to 10 as a dispersing medium. The amount of zinc oxide or titanium dioxide is generally not more than 99.9% by mass, preferably not more than 90% by mass, and more preferably not more than 85% by mass of the total mass of the dispersion.

The dispersion of the present invention may contain other additives according to need. Examples of such additives include dispersants other than the above polyether-modified silicone, diluents, and stabilizers. The amount of the additives may appropriately be selected as long as the amount of the zinc oxide or titanium dioxide does not fall to less than 60% by mass of the whole mass of the dispersant, and the dispersibility of the zinc oxide or titanium dioxide is maintained.

Ingredients other than zinc oxide or titanium dioxide contained in a dispersion are an isoparaffin having the average polymerization degree of 3 to 10 (dispersion medium), the dispersant as mentioned above, optional additives added according to need, and so on. The amount of zinc oxide or titanium dioxide is not less than 60% by mass, and preferably not less than 70% by mass. Therefore, the amount of the isoparaffin is not more than 40% by mass, and preferably not more than 30% by mass. By reducing the isoparaffin amount corresponding to the amount of dispersants or additives to be added, the total pigment concentration is adjusted so that the pigment concentration may keep a level not less than 60% by mass of the dispersion.

The dispersion may be produced by any known methods. For example, the dispersion of the present invention may be produced by dispersing an appropriate mixture with an apparatus such as a bead mill, a ball mill, a high-pressure homogenizer, a stirring homogenizer, an ultrasonic homogenizer, and a wet jet mill. These apparatuses can be freely selected from the viewpoints of a degree of distribution, production efficiency, and so on.

The dispersion of the present invention can be blended in cosmetics, such as sunscreens or foundations. The cosmetic thus-obtained is free of volatile silicones, or even if it contains a certain volatile silicone, its amount is very small. Thus, the cosmetic provides excellent feeling of use.

### Example

Hereinafter, “part(s)” means part(s) by mass, and “%” means % by mass, unless otherwise indicated.

### Example 1

A bottle (mayonnaise bottle) was charged with 70 parts of hydrophobically-treated zinc oxide (FINEX-30-LPT, product of Sakai Chemical Industry Co., Ltd; produced by treating the surface of zinc oxide with organopolysiloxane), 22 parts of an isoparaffin (PearLeam 4, product of NOF Corporation), 8 parts of a polyether-modified silicone (KF-6038, product of Shin-Etsu chemicals Co., Ltd., HLB=3.0), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Example 2

A bottle (mayonnaise bottle) was charged with 70 parts of hydrophobically-treated zinc oxide (FINEX-30W-LP2, product of Sakai Chemical Industry Co., Ltd; produced by treating the surface of zinc oxide with silica and an organopolysiloxane), 22 parts of an isoparaffin (PearLeam 4, product of NOF Corporation), 8 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Example 3

A bottle (mayonnaise bottle) was charged with 60 parts of hydrophobically-treated titanium dioxide (STR-100A-LPT, product of Sakai Chemical Industry Co., Ltd; produced by treating the surface of zinc oxide with silica, alumina, and organopolysiloxane), 28 parts of an isoparaffin (PearLeam 4, product of NOF Corporation), 12 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a titanium dioxide dispersion.

### Comparative Example 1

A bottle (mayonnaise bottle) was charged with 60 parts of hydrophobically-treated zinc oxide (the same material as the zinc oxide used in Example 1), 32 parts of isononyl isononanoate (CETIOL ININ, product of Cognis Japan), 8 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 2

A bottle (mayonnaise bottle) was charged with 60 parts of hydrophobically-treated zinc oxide (the same material as the zinc oxide used in Example 1), 32 parts of trioctanoin (EMALEX, product of Nihon Emulsion Co., Ltd.), 8 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 3

A bottle (mayonnaise bottle) was charged with 60 parts of hydrophobically-treated zinc oxide (the same material as the zinc oxide used in Example 1), 32 parts of liquid paraffin (Sumoil P-80, product of Matsumura Oil Co., Ltd.), 8 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 4

A bottle (mayonnaise bottle) was charged with 60 parts of hydrophobically-treated zinc oxide (the same material as the zinc oxide used in Example 1), 40 parts of an isoparaffin (PearLeam 4, product of NOF Corporation), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 5

A bottle (mayonnaise bottle) was charged with 60 parts of hydrophobically-treated zinc oxide (the same material as the zinc oxide used in Example 1), 32 parts of an isoparaffin (PearLeam 4, product of NOF Corporation), 8 parts of a polyether-modified silicone (KF-6012, product of Shin-Etsu chemicals Co., Ltd., HLB=7.0), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 6

A bottle (mayonnaise bottle) was charged with 60 parts of hydrophobically-treated titanium dioxide (the same material as the titanium dioxide used in Example 3), 28 parts of isopropyl palmitate (Crodamol IPP, product of Croda), 12 parts of polyhydroxystearic acid (Solsperse 3000, product of Lubrizol Corporation), and 100 parts of φ0.5-mm zirconia beads, and the ingredients were mixed. The mixture was processed with a paint shaker (Red Devil Inc.) for 1 hour. The beads were separated from the mixture, to give a titanium dioxide dispersion.

### Comparative Example 7

A bottle (mayonnaise bottle) was charged with 50 parts of hydrophobically-treated titanium dioxide (the same material as the titanium dioxide used in Example 3), 38 parts of isopropyl palmitate (Crodamol IPP, product of Croda), 12 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a titanium dioxide dispersion.

### Comparative Example 8

A bottle (mayonnaise bottle) was charged with 70 parts of hydrophobically-treated zinc oxide (the same material as the zinc oxide used in Example 2), 22 parts of decamethylcyclopentasiloxane (KF-995, Shin-Etsu chemicals Co., Ltd.), 8 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a zinc oxide dispersion.

### Comparative Example 9

A bottle (mayonnaise bottle) was charged with 60 parts of hydrophobically-treated titanium dioxide (the same material as the titanium dioxide used in Example 3), 28 parts of decamethylcyclopentasiloxane (KF-995, Shin-Etsu chemicals Co., Ltd.), 12 parts of a polyether-modified silicone (KF-6038, product of Shin-Etsu chemicals Co., Ltd.), and 100 parts of φ0.5-mm zirconia beads, and the ingredients were mixed. The mixture was processed with a paint shaker (Red Devil Inc.) for 1 hour. The beads were separated from the mixture, to give a titanium dioxide dispersion.

### [Viscosity]

The dispersion was put into a 50-ml screw bottle, and the viscosity of the dispersion was measured at 60 seconds after start of rotation using a Brookfield viscometer (made by Tokyo Keiki Inc.), the type of the rotor being No. 3. The data of the dispersion viscosity (25° C.) after dispersion is listed in Table 1.

As recognized from the results in Table 1, the dispersions of Examples 1 to 3, which contained the isoparaffin as a medium had low viscosity, and did not form gel even if the concentration of hydrophobic zinc oxide was as high as 70% by mass, or the concentration of titanium dioxide was as high as 60% by mass.

On the contrary, when isononyl isononanoate, trioctanoin, or liquid paraffin was used as the dispersion medium, gel was formed after dispersing process even if the concentration of hydrophobic zinc oxide was 60% by mass in the dispersant. Gelation was also observed even under concentration of hydrophobic zinc oxide of 60% by mass in the case where a polyether-modified silicone was not used as the dispersant, or the case where a polyether-modified silicone with an HLB value of 7.0 was used as the dispersant (comparative examples 4 and

The dispersion disclosed in the Patent Document 1, which contained titanium dioxide, isopropyl palmitate (Crodamol IPP, product of Croda), and polyhydroxystearic acid (Solsperse 3000, product of Lubrizol Corporation) cause gelation under the concentration of the hydrophobic zinc oxide was 60% by mass in the dispersant (comparative example 6). Furthermore, when a polyether-modified silicone with an HLB value of 3.0 was used instead of the polyhydroxystearic acid in the comparative example 5 as a dispersant, gelation was observed under the concentration of hydrophobically-treated titanium dioxide of 50% by mass (comparative example 7).

When the decamethylcyclopentasiloxane, which have been frequently used as volatile oil, was used, the dispersion was gelled under the concentration of the hydrophobically-treated zinc oxide of 70% by mass, or the concentration of the hydrophobically-treated titanium dioxide of 60% by mass.

Thus, as understood from the above results, the isoparaffin as a dispersion medium serves to enhance dispersibility of pigments such as titanium dioxide or zinc oxide in a high pigment concentration. Particularly, the dispersion containing decamethylcyclopentasiloxane, which has widely been used up to now, could not attain such a high level of the pigment concentration, as demonstrated in comparative examples 8 and 9. It is unexpected that only the present invention could provide a dispersion with such a high pigment concentration that a common dispersion medium, decamethylcyclopentasiloxane, cannot attain.

TABLE 1

| | | Viscosity |
|---|---|---|
| | | (mPa · s/25° C.) |
| Example | 1 | 1423 |
| | 2 | 639 |
| | 3 | 1730 |
| Comparative | 1 | Gelled |
| Example | 2 | Gelled |
| | 3 | Gelled |
| | 4 | Gelled |
| | 5 | Gelled |
| | 6 | Gelled |
| | 7 | Gelled |
| | 8 | Gelled |
| | 9 | Gelled |

1. A dispersion comprising:
zinc oxide or titanium oxide;
an isoparaffin having the average polymerization degree of 3 to 10; and
a polyether-modified silicone having an HLB value of 2 to 5, as a dispersant;
the amount of the zinc oxide or titanium oxide being not less than 60% by mass of the whole amount of the dispersion.

2. The dispersion according to claim 1,
wherein the amount of the zinc oxide or titanium oxide is preferably not less than 70% by mass of whole amount of the dispersion.

3. The dispersion according to claim 1,
wherein the dispersion preferably contains 2 to 15% by mass of the polyether-modified silicone relative to the whole amount of the dispersion.

4-6. (canceled)

7. The dispersion according to claim 2,
wherein the dispersion preferably contains 2 to 15% by mass of the polyether-modified silicone relative to the whole amount of the dispersion.

8. The dispersion according to claim 1,
wherein the zinc oxide or titanium oxide has a surface treated with at least one of the group consisting of silica, alumina, alkyl silanes, and organopolysiloxanes.

9. The dispersion according to claim 2,
wherein the zinc oxide or titanium oxide has a surface treated with at least one of the group consisting of silica, alumina, alkyl silanes, and organopolysiloxanes.

10. The dispersion according to claim 3,
wherein the zinc oxide or titanium oxide has a surface treated with at least one of the group consisting of silica, alumina, alkyl silanes, and organopolysiloxanes.

11. The dispersion according to claim 4,
wherein the zinc oxide or titanium oxide has a surface treated with at least one of the group consisting of silica, alumina, alkyl silanes, and organopolysiloxanes.

12. The dispersion according to claim 1,
wherein the zinc oxide or titanium oxide preferably has an average primary particle diameter of 100 nm or smaller.

13. The dispersion according to claim 2,
wherein the zinc oxide or titanium oxide preferably has an average primary particle diameter of 100 nm or smaller.

14. The dispersion according to claim 3,
wherein the zinc oxide or titanium oxide preferably has an average primary particle diameter of 100 nm or smaller.

15. The dispersion according to claim 7,
wherein the zinc oxide or titanium oxide preferably has an average primary particle diameter of 100 nm or smaller.

16. A cosmetic composition comprising a dispersion according to claim 1.

17. A cosmetic composition comprising a dispersion according to claim 2.

18. A cosmetic composition comprising a dispersion according to claim 3.

19. A cosmetic composition comprising a dispersion according to claim 7

20. A cosmetic composition comprising a dispersion according to claim 8.
